(19) United States
(12) Patent Application Publication
(10) Pub. No.: US 2024/0225615 A1
(43) Pub. Date: Jul. 11, 2024

(54) FAECAL SAMPLING DEVICE AND METHOD

(71) Applicant: HEALTH FIRST LABORATORIES LTD., Harrow, Middlesex (GB)

(72) Inventor: Howard Chandler, Avoca Beach, New South Wales (AU)

(21) Appl. No.: 18/558,989

(22) PCT Filed: May 5, 2022
(86) PCT No.: PCT/AU2022/050421

§ 371 Date: Nov. 3, 2023

(30) Foreign Application Priority Data
May 6, 2021 (AU) 2021901350

(51) Int. Cl. A61B 10/00; A61B 17/34

(52) U.S. Cl. CPC A61B 10/0038; A61B 17/34

(57) ABSTRACT

A sampling device, assembly and method to obtain a biological sample for a patient including a faccal sample. The sampling device (10) comprises an elongate guide member body (20) having an open end (21) and a leading end (22); the guide member body having an inner wall (23) which defines a channel (30) extending from an opening (25) in the leading end to the open end; the inner wall defining; a main region of the channel (31), the main region extending from the open end towards the leading end; a leading region of the channel (33); and one or more shoulders (32) between the main region of the channel and the leading region of the channel.

### TECHNICAL FIELD

The present disclosure relates to a sampling device, assembly and method which may be used to obtain a biological sample for a patient including a faecal sample.

### BACKGROUND

Faecal samples can be used, amongst other things, for colorectal cancer screening. When detected early, death from colorectal cancer should be preventable in many cases. Unfortunately, due to the lack of acceptable collection methods, colorectal cancer remains one of the top three most common causes of cancer deaths.

One means for early detection of colorectal cancer is by screening for traces of blood released into faecal matter by the developing cancer or its precancerous polyps. Patients found to have positive results are then sent for colonoscopy or surgery and the cancerous lesions are removed.

Despite the proven value of faecal screening for this condition, persuading the at-risk population to collect and submit a faecal sample remains a challenge. This leads to the poor compliance that limits the value of bowel cancer screening programs.

Before the availability of Faecal Immunochemical Tests (FITs) for screening, guaiac based Faecal Occult Blood Tests (FOBTs) were used to detect blood in the stool. As FOBTs were subject to numerous interferences from foodstuffs and required several days of dietary restrictions and multiple test samples, compliance by test subjects was poor. Nevertheless, FOBT tests were proven to save lives and accordingly recommended by some cancer control agencies.

To address the poor FOBT compliance issue, physicians tended to perform digital-rectal examination (DRE), to obtain a small amount of faeces which was then spread on a guaiac card (for example, Haemoccult) and developed on the spot to test for the presence of any blood. Although this solved the compliance issue, lack of any dietary restrictions and the possible contamination from anal bleeding could cause false positive results. For these reasons DRE testing was not recommended by many cancer control agencies.

FOBTs have now been replaced by the more sensitive and specific immunoassays such as FITs. FITs only require one stool to be sampled and are specific for blood from the large intestine (bowel), which is where most colorectal cancers originate. FITs typically use a kit with a probe or stick for the patient to collect a sample from stool collected on toilet paper, or from a paper sling suspended over the toilet bowl. The probe with stool sample is added to a stabilising buffer in a tube and sent to a lab for analysis, where the result is reported as micrograms of haemoglobin (Hb) per gram of stool, i.e., ug Hb/g of stool. The actual mass of stool collected depends on the density and the consistency of the stool and the diligence of the collector (in many cases, aging collector), so only semi-quantitative results are possible.

Despite their many advantages, poor compliance remains a major problem with FIT screening. This may be due both to the requirement for intimate faecal contact and the unpleasant prospect of a possible cancer diagnosis.

The disclosure herein may address the poor compliance with FIT testing by providing physicians with a simple means for collecting a stool sample for FIT testing during a patient consultation. Patients testing positive may then be guided as to next steps, typically a colonoscopy, during the consultation.

Blood from haemorrhoids and anal fissures may contaminate the spontaneously passed stools sampled by existing FITs and FOBTs, potentially leading to positive test results which can be associated with bowel abnormality and would in those cases lead to overuse of expensive colonoscopies. Thus, there is a need for a sampling device designed to reduce positive test results from blood originating from haemorrhoids, anal fissures or any other source of surface bleeding.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

Aspects of this disclosure may provide a faecal sampling device designed to reduce positive test results from haemorrhoids, anal fissures and any bleeding caused by the sampling device itself. Samples may be taken from the rectum, before the faeces passes through the anus, and as such, samples may be less likely to be contaminated by blood from haemorrhoids, anal fissures or damage to the anus. Furthermore, aspects of this disclosure may provide a faecal sampling device designed to minimise tissue damage induced anal bleeding which may interfere with test results.

In one aspect there is provided a faecal sampling device comprising
an elongate guide member body having an open end and a leading end;
the guide member body having an inner wall which defines a channel extending from an opening in the leading end to the open end;
the inner wall defining:
   a main region of the channel, the main region extending from the open end towards the leading end;
   a leading region of the channel; and
   one or more shoulders between the main region of the channel and the leading region of the channel.

In another aspect there is provided a sampling assembly comprising:
a sampling device including an elongate guide member body having an open end and a leading end;
the guide member body having an inner wall which defines a channel extending from an opening in the leading end to the open end;
the inner wall defining:
   a main region of the channel, the main region extending from the open end towards the leading end;
   a leading region of the channel; and
   one or more shoulders between the main region of the channel and the leading region of the channel; and
a sampling member including a shaft extending from a handling end to a sample capture end, the sampling member configured to extend through the channel of the guide member.

The guide member may be inserted into the anal canal of the subject by a physician such that the opening of the guide member reaches the rectum of the patient, where a faecal sample may be taken.

The guide member may extend along a longitudinal axis from the open end to the leading end. The channel may be substantially centred or may extend along an axis which is offset relative to the longitudinal axis.

The guide member may further comprise a flange member. The flange member may be integral with or connected to the guide member body. The flange member typically extends away from, and radially outwardly from, a junction with the guide member body to an outer rim to form a funnel. The flange member may be configured, in use, to sit outside of the anus with an outer surface of the flange member abutting the skin around the anus. As such, the flange member may limit how far the guide member body may be inserted into the anal canal of a patient.

In one embodiment, the flange member is positioned at or close to the open end of the guide member. Alternatively, the flange member may be spaced from the open end towards the leading end of the guide member.

The sampling device may further include a handle. The handle may extend from part of the flange member. Typically, the handle extends from a part of the outer rim of the flange member although it may be connected to any region of the flange member. In another embodiment, if the sampling device does not include a flange member, the handle may be connected to the guide member body at or adjacent to the open end.

The handle is typically configured to extend at an angle orthogonal to a main axis of the guide member body. In this arrangement, when the guide member body is inserted into an anal canal of a patient, the handle may sit substantially flush with the skin of the patient. Optionally, the handle further comprises one or more gripping features to allow a user to securely hold the handle during use of the sampling device.

The guide member body may further comprise a cap to cover or seal the opening in the leading end. The cap may be moveable from a first covering configuration to a second released configuration.

The cap may be fully released from the guide member body. In this embodiment, the release may occur once the guide member body is optimally positioned within the rectum. Alternatively, the cap may be partially released in which embodiment, part of the cap may be still connected to the guide member body adjacent the opening, but not covering the opening.

In one embodiment, the cap comprises a plug which sits partially within the leading region of the channel. In this embodiment, the plug may be dislodged from within the channel and through the opening in the guide member body by pressure exerted on it from the sampling member as the sampling member is moved through the channel of the guide member body.

The cap may be made from any material suitable for medical use. For example, the cap may comprise a wax or other non-absorbent material. The cap may be made from a resorbable or degradable material such that if it is fully released into the rectum, it will degrade or be resorbed over time. The cap may comprise a thin film-like member which sits across the opening. The cap may comprise a thin membrane such as an aluminium foil membrane which may be readily perforated without the need for full removal.

Preferably, an external surface of the guide member body is smooth and devoid of sharp edges, corners or protrusions such as to prevent damage to the mucosal walls of the anal cavity or rectum of a patient during insertion, as well as facilitating a smoother insertion. The outer wall of the leading end of the guide member body may also be curved or smoothly tapered to provide an atraumatic leading end. In another embodiment, the guide member body may include a low friction coating on its outer surface to enable a smooth movement through the anal cavity and avoid damage to the surrounding mucosal walls.

The outer wall may include other coatings including antimicrobial coatings, wherein the coating is selected such that it does not interfere with sample taking or sample testing.

A shoulder defined by the inner wall may provide an abutment surface. The abutment surface may extend substantially perpendicular to the main axis of the guide member body. In other embodiments, however, the abutment surface may comprise an angled surface of the inner wall extending from a junction with the inner wall which defines the main region of the channel to a junction with the inner wall which defines the leading region of the channel. The abutment surface of this embodiment typically extends inwardly to decrease the cross sectional area of the channel. Where the channel is substantially circular in cross section, the inwardly extending abutment surface decreases the diameter of the channel towards the leading region of the guide member body.

A cross sectional area of the leading region of the channel is typically less than the cross sectional area of the main region of the channel. The larger dimensioned channel of the main region may allow for ease of insertion of a sampling member into the guide member body. The dimensions of the leading region of the channel are typically such that part of the sampling member of the second aspect tightly fits therein. In this embodiment, a portion of the sampling member may have a larger diameter than the leading region of the channel and may be relatively compressible such that pressure from the inner wall defining the leading region of the channel compresses the portion at a pressure that still allows the portion to move through the leading region of the channel and beyond the opening in the leading end of the guide member body. Particularly, the compressible portion of the sampling member may be the sample capture end.

In another embodiment, the inner wall defining the leading region of the channel may include one or more restriction regions. The restriction regions may be regions of reduced diameter of the channel. The restriction regions may include a number of features of the sidewall including, but not limited to, ridges or bumps which extend into the channel. In one embodiment, the inner wall includes one or more inwardly protruding annular rims. In a further embodiment, the restriction region is achieved by an O-ring.

The cross-sectional area of the main region of the channel may taper along its length towards the one or more shoulders. Alternatively, the main region of the channel may be substantially uniform in cross sectional area along its length.

The guide member body may be of any length suitable for obtaining a faecal sample from a subject as required. For the purposes of obtaining a faecal sample from the rectum of a subject, the guide body may have a length from the leading end to the open end of between about 50 mm and 120 mm. In one embodiment, the length of the guide may be about 110 mm. The length of the guide member body may be selected to place the leading end at an optimal position within the rectum and far enough into the anal cavity to avoid picking up any contaminant such as blood from haemorrhoids or anal fissures, which could provide a false positive result.

The length of the leading region of the channel may be between about 1 mm and 15 mm. In some embodiments, the length of the leading region of the channel is approximately 1 mm, 5 mm, 6 mm, 7 mm, 8 mm, 9 mm or 10 mm.

A cross-sectional dimension of the leading region of the channel may be less than about 7 mm, less than about 6 mm, less than about 5 mm, less than about 4 mm, less than about 3 mm or less than about 2 mm. In one embodiment, the leading region of the channel is circular in cross-section and has a diameter of approximately 3 mm. The leading end opening may be dimensioned to provide a tight friction fit for at least a portion of the sampling member such that it controls the amount of faecal material collected by the sampling device through a process of “wiping” as the member is moved through the opening. In this regard, the size of the opening is dimensioned to be slightly less that the cross sectional dimension of at least part of the sampling device.

The guide member body may comprise a single integrally molded member. Alternatively, the guide member body may comprise two or more components. The two components may be coupled or molded together. For example, the guide member body may comprise an outer housing which is configured to receive and house an inner housing. In this embodiment, the inner housing includes the features of the inner wall which define the channel and the outer housing is adapted to be in contact with a subject when the guide member body is in use.

The sampling device as described may be made from any material suitable for medical use. Examples include a plastic or silicone based material.

The sampling member typically comprises a relatively rigid elongate shaft and an enlarged sample capture end. The sample capture end may include any suitable material to collect sufficient faeces for testing. In one embodiment the sample capture end comprises a natural or synthetic flocked material. In another embodiment the sample capture end may comprise natural or synthetic material spun around the sample capture end similar to a standard cotton tip. In another embodiment, the sample capture end may comprise a non-woven material. The sample capture end may further include a cellulosic material.

The channel of the guide member body is configured to receive the sampling member. The dimensions of the main region of the channel may be of sufficient size to allow the relatively free insertion of the sampling member without the sampling member necessarily touching the sidewall defining the channel. The one or more shoulders of the sidewall typically cause a narrowing of the channel towards the leading region of the channel. As the sample capture end of the sampling member is advanced towards the leading region of the channel, the sample capture end may come into abutment with the sidewall.

The sample capture end may relatively tightly fit through the leading region of the channel before being pushed out of the channel through the open end. The shaft of the sampling member may be of a sufficient length that when the sample capture end extends beyond the open end of the guide member body, the handling end extends sufficiently beyond the open end of the guide member body such that a user may readily manipulate the sampling member. For example, once the sample capture end is pushed beyond the open end, a user may manipulate the shaft at the handling end to cause the sample capture end to rotate and/or move within the rectum to pick up sufficient faecal matter for testing. The handling end of the sampling member may include a finger grip member to allow ease of use.

The sampling member may have a length of between about 50 mm and about 150 mm. The sample capture end may have a length of between about 1 mm and about 20 mm. In one embodiment, the length of the sampling member is about 150 mm and the length of the sample capture end is about 5 mm.

The sampling member may further include a collar positioned on part of the shaft. The collar may be positioned at a pre-determined distance from the sample capture end. In some embodiments, the collar is configured to abut with the one or more shoulders of the sidewall of the guide member body when the sampling member is inserted therein. In this embodiment, abutment of the collar against the one or more shoulders limits the distance that the sample capture end of the sampling member may be extended through and beyond the open end of the guide member body.

The shaft may have a cross sectional dimension of between about 1.0 mm and 3 mm. The sample capture end of the sampling member may have a cross sectional dimension which is larger than a cross sectional diameter of the shaft. For example, the cross sectional diameter of the sample capture end may be, for example, approximately 2 mm to 4 mm. In one embodiment where the shaft has, for example, a cross sectional diameter of 2 mm, the capture end may have a cross sectional diameter of 2.2 mm. The collar may have a cross sectional dimension of between about 3 mm and about 5 mm.

In a further aspect there is provided a method for obtaining a faecal sample from a subject, the method comprising:
inserting an elongate guide member body into the rectum of a subject, the guide member body comprising:
   an open end and a leading end;
   an inner wall which defines a channel extending from an opening in the leading end to the open end, the inner wall defining:
      a main region of the channel, the main region extending from the open end towards the leading end;
      a leading region of the channel; and
      one or more shoulders disposed between the main region of the channel and the leading region of the channel;
inserting a sampling member into the open end of the guide member, the sampling member having a shaft, a handling end and a sample capture end wherein at least part of the sample capture end has a cross-sectional area greater than the cross-sectional area of the shaft;
advancing the sampling member through the channel until the sample capture end extends beyond the opening in the leading end of the guide member body;
capturing a faecal sample on the sample capture end of the member;
withdrawing the sampling member, wherein withdrawal of the sample capture end through the opening and the leading region channel of the guide member body removes any excess faecal sample from the sample capture end; and
further withdrawing the sampling member through the main region of the channel and from the open end of the guide member body.

The relative dimensions of the sample capture end and the opening and/or the leading region of the channel provide a means to provide an optimal quantity of faecal sample. Other means such as the restriction regions described above may further meter the amount of sample carried on the sample capture end.

In a further aspect there is provided a method of testing a faecal sample, the method comprising:
the aforementioned method of obtaining a faecal sample from a subject;
testing the faecal sample for the presence of blood using a Faecal Immunochemical Test (FIT) strip or other suitable immunoassay method.

In yet another aspect there is provided a method of testing a faecal sample, the method comprising:
the aforementioned method of obtaining a faecal sample from a subject;
testing the faecal sample from the subject using a Faecal occult blood test (FOBT).

### DESCRIPTION OF THE DRAWINGS

FIG. 1 is a cross sectional side view of an embodiment of the sampling device;

FIG. 2 is a side, partially cross sectional side view of an embodiment of the sampling assembly comprising a sampling member inserted through the sampling device;

FIG. 3 is a schematic view of an embodiment of a sampling member of the disclosure;

FIG. 4A is a side, partially cross sectional view of an embodiment of the assembly comprising a further embodiment of the sampling member inserted through the sampling device;

FIG. 4B is a side view of the embodiment of the sampling member of FIG. 4A; and

FIG. 4C is a magnified view of the sample capture end of the sampling member of FIG. 4B.

FIG. 5A is a schematic view of an embodiment of the disclosure with the sampling member extending therethrough;

FIG. 5B is a side view of the sampling member shown in FIG. 5A;

FIG. 6 is a cross sectional view of a further embodiment of a guide member according to the present disclosure;

FIGS. 7A and 7B are schematic views of further embodiments of the guide member;

FIG. 8 is a side cross sectional view of another embodiment of the guide member;

FIG. 9 is a further cross sectional side elevated view of the guide member of FIG. 7;

### DESCRIPTION OF EMBODIMENTS

The singular forms “a”, “an” and “the” include plural referents unless the context clearly dictates otherwise.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Sampling device 10 as shown in the figures comprises guide member body 20 which has an open end 21, a leading end 22 and an inner wall 23 which defines a channel 30 extending from an opening 25 in the leading end 22 to the open end 21. The inner wall 23 defines a main region 31 of the channel 30 which extends from open end 21 towards leading end 22 and a leading region 33. Guide member body 20 further includes one or more shoulders 32 between main region 31 and leading region 33 of channel 30.

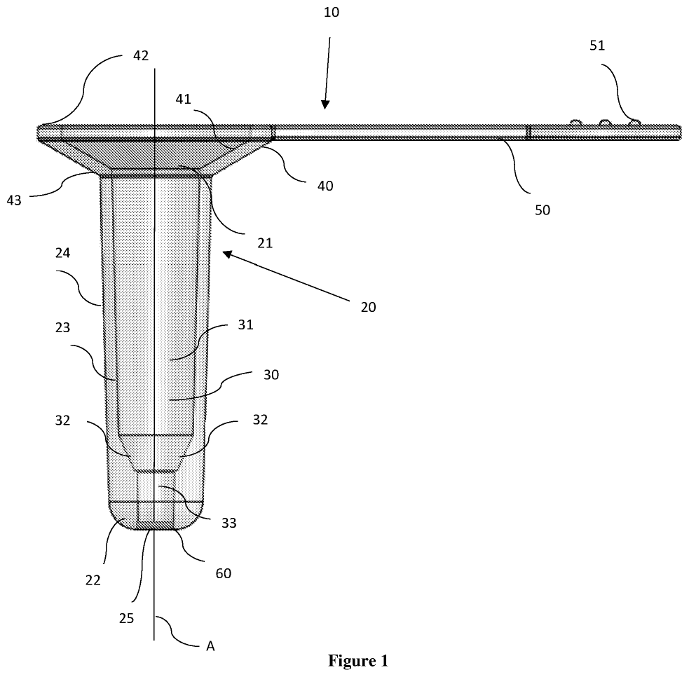

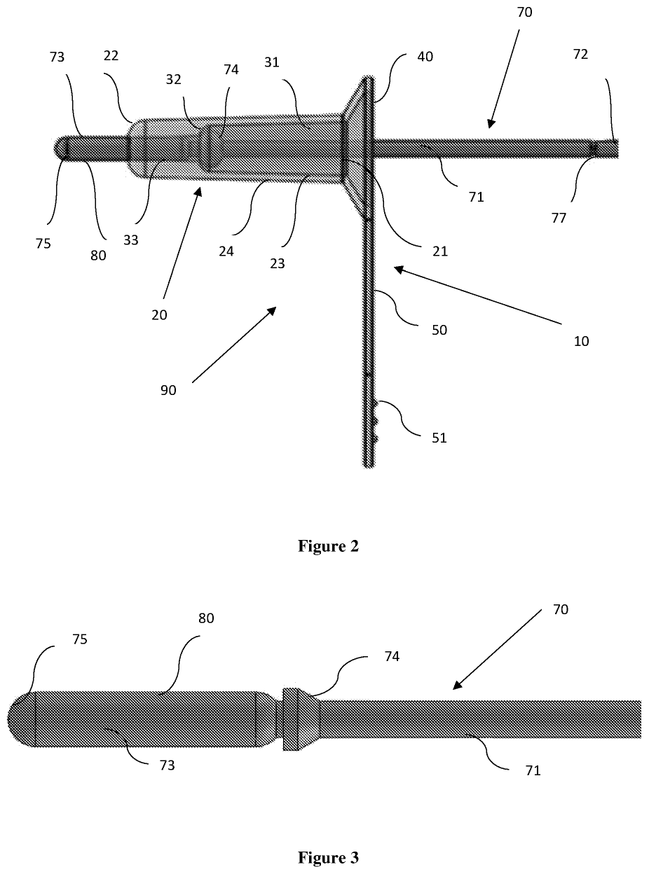

As shown in, for example, FIGS. 1 and 2, leading end 22 of guide member body 20 is slightly curved towards opening 25 to provide an atraumatic leading tip.

An outer wall 24 of guide member body 20 as shown is smooth and devoid of sharp components such as any sharp edges, corners or protrusions.

Sampling device 10 further includes flange member 40 extending away and radially outwardly from a junction with open end 21 to an outer rim 42. An inner surface 41 of flange member 40 defines a conical receptacle. In use, guide member body 20 is inserted via the anal cavity into the rectum of the patient. Due to the increased diameter of flange member 40, it sits outside of the patient's body with an outer surface 43 resting against the skin around the anus. In this position, a user may readily insert further medical equipment such as sampling member 70 into the conical receptacle defined by inner surface 41 of flange member 40.

Sampling device 10 may further comprise a handle 50. Handle 50 may facilitate insertion of guide member 20 via the anal canal into the rectum of the patient as well as aiding in its removal. Handle 50 may also be used to manoeuvre the guide member within the rectum to optimise the capture of a test sample. As depicted, handle 50 extends from a junction with outer rim 42 of flange member 40. In embodiments where the device does not have a flange member, handle 50 may instead extend from the guide member body 20, at or adjacent open end 21.

Handle 50 extends in a plane substantially orthogonal to main axis A of the sampling device 10 which allows it to sit adjacent to or against the skin of the patient when guide member body 20 is inserted into the anal cavity. Handle 50 also includes grip features 51 comprising any expanded portions, textured materials, ridges, protrusions, notches or depressions adapted to facilitate the manual manipulation of handle 50.

As shown in FIG. 1, guide member body 20 may be sealed across opening 25 by a cap or plug 60. Other forms of caps are envisaged and the present depiction is an example of one suitable type of cap only.

Plug 60 seals opening 25 during insertion of the guide into the anal cavity of the patient and helps to prevent contamination of the channel 30 of the guide member body 20 as it is inserted into the patient.

In one example, plug 60 is either partially or fully removable by pushing sampling member 70 against the plug. This dislodges the plug which is typically made from a biocompatible material such as a wax such that it is safe to leave within the rectum (if fully dislodged). In other examples not depicted, plug 60 may remain hingedly attached to guide member body 20.

The one or more shoulders 32 defined by the inner wall 23 may take on any suitable form which presents an abutment feature. The shoulders 32 may be substantially perpendicular to the length of channel 30. As depicted in the exemplary embodiments provided, shoulders 32 taper towards the leading region 33 of the channel 30. In this example, the taper guides a sampling member 70 from the main region 31 of the channel 30 into the leading region 33 of the channel 30.

As shown in, for example FIG. 1, a cross-sectional dimension of leading region 33 of the channel 30 is less than a cross-sectional dimension of the main region 31 of the channel 30. The relatively narrow cross-sectional dimensions of the leading region 33 of the channel 30 contributes to the removal of excess faecal sample from the sample capture end 73 of the sampling member 70 as discussed further below.

Sampling device 10 is particularly suited to obtaining a faecal sample from the rectum of a subject, although it should be understood that it may also be suitable for obtaining other sample types from a subject via a different cavity.

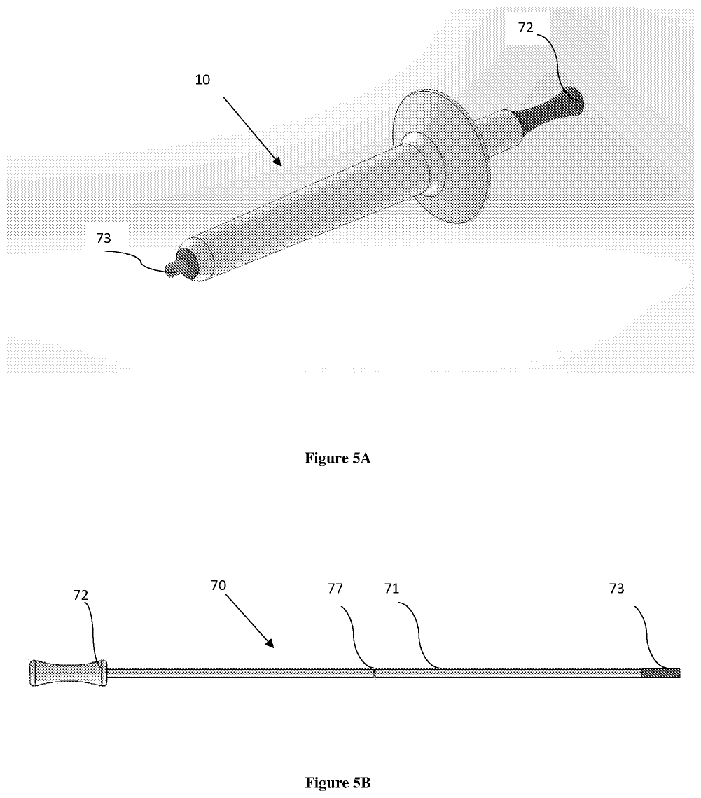

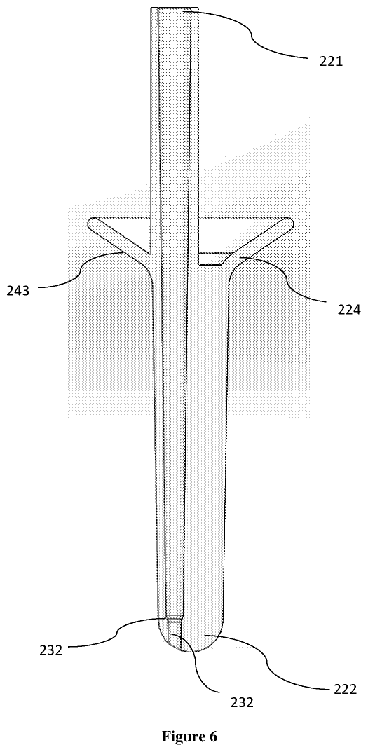

In yet a further aspect, as shown, for example, in FIGS. 5 to 9, sampling device 210 comprises an elongate guide member body 220 having an open end 221 and a leading end 222. The guide member body 220 further comprises an outer wall 224 having a sloped, rounded or hemispherical surface at the leading end 222, the outer wall 224 surrounded by a collar 240 at a proximal end 226 of the outer wall 224. Alternatively, as shown in FIG. 6, the collar is positioned spaced from open end 221. Guide member body 220 further comprises an inner wall 223 which defines a channel 230 extending from an opening 225 in the leading end 222 to the open end 221. Open end 221 is spaced from the collar 240 by a grip portion 228 of the inner wall 223 which extends beyond the proximal end 226 of the outer wall 224.

As shown, for example, in FIGS. 5, 6, 7A and 7B, leading end 222 of guide member body 220 is slightly curved towards longitudinal axis A thereby providing an atraumatic leading tip. In this example, channel 230 and opening 225 are along an axis which is longitudinally offset from longitudinal axis A. In these examples, opening 225 is formed in the curved part of the leading end.

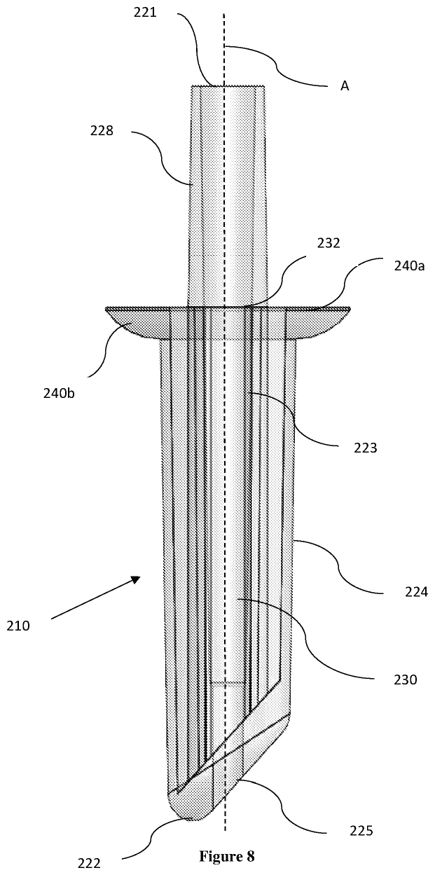

In another embodiment, as shown, for example, in FIG. 8, leading end 222 of guide member body 220 forms an oblique end surface relative the longitudinal axis A of the elongate guide member body 220. Oblique leading end 222 may be rounded and gently profiled to provide for an atraumatic leading tip offset from the longitudinal axis A of the elongate guide member body 220. In this example, a centre of opening 225 is generally aligned with axis A and opening 225 is provided oblique to axis A. The opening may also be longitudinally offset as described above.

The length of the inserted portion, being the length of the outer wall from the leading end 222 to collar 240 is preferably sufficient to clear any blood around the anal sphincter such that stool which is more likely uncontaminated by blood can be sampled. Accordingly, a length from the collar to the leading end may be about 20 mm to 90 mm, about 25 mm to 65 mm, about 30 mm to 60 mm, about 40 mm to about 50 mm or about 45 mm.

Sampling device 210 further includes collar 240 extending radially outwardly from outer wall 224. In use, guide member body 220 is inserted via the anus into the rectum of the patient. Collar 240 controls the depth of insertion of the guide. Due to the increased diameter of collar 240, it sits outside of the patient's body with an outer surface 243 resting against the skin around the anus. In this position, a user may readily insert further medical equipment such as sampling member 270 into the channel 230 defined by inner wall 223.

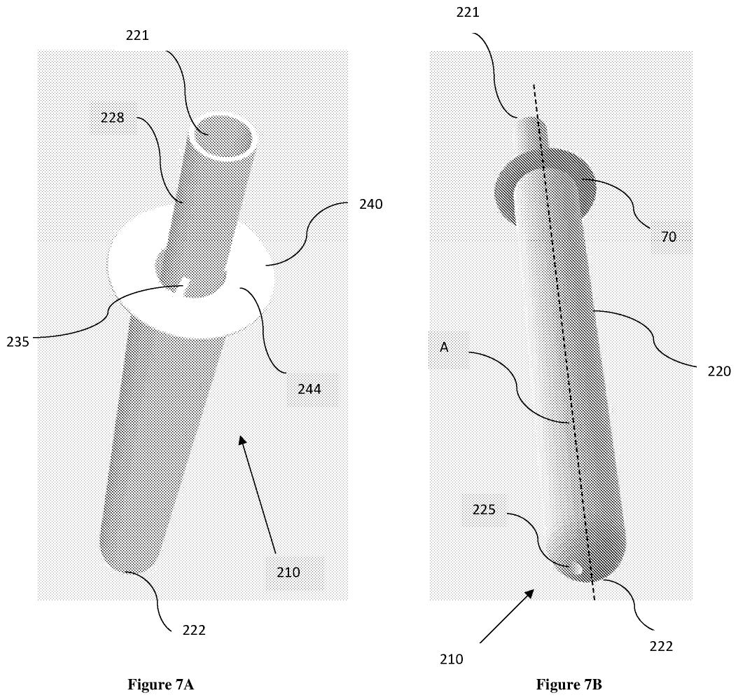

As shown, for example, in FIGS. 7 to 9, open end 221 is spaced from the collar 240 by a grip portion 228 of the inner wall 223 which extends beyond the proximal end 226 of the outer wall 224. Grip portion 228 allows a user to control sampling device 210 during insertion, sampling and removal. Grip portion 228 allows the user to manoeuvre the guide member within the rectum to optimise the capture of a test sample. The exterior surface of grip portion 228 may be smooth. Alternatively it may further comprise grip features, not shown, comprising any expanded portions, textured materials, ridges, protrusions, notches or depressions adapted to facilitate the manual manipulation of the grip portion 228.

As shown in for example FIG. 8, collar 240 comprises an upper surface 240a and a lower surface 240b wherein the lower surface is configured to contact the skin around the anus. Lower surface 240b may be curved upwardly away from outer wall 224 of elongate guide member body 220. Upper surface 240a may provide an abutment surface for the hand of the user during insertion, sampling and removal.

Sampling device 210 may be integrally formed. Device 210 may be formed such that inner wall 223 is fused with outer wall 224 along the length of the outer wall 224.

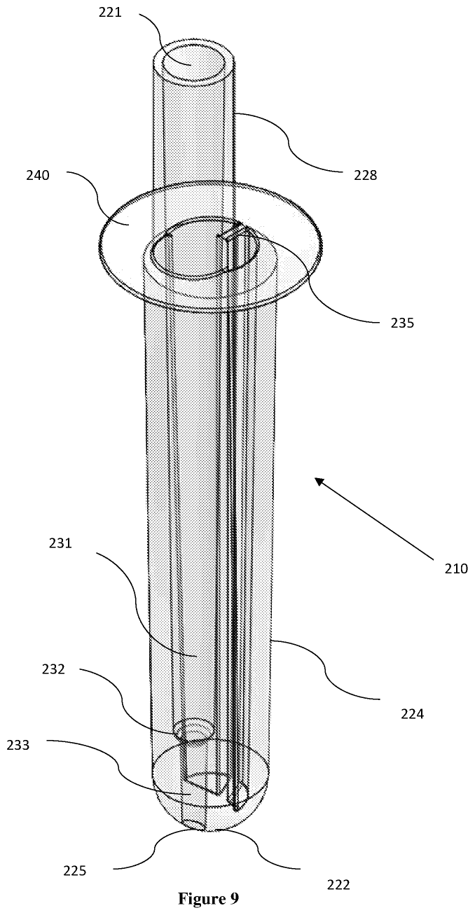

Alternatively, sampling device 210 may be formed such that there is a gap provided between inner wall 223 and outer wall 224 along the length of the outer wall 224. In such case, inner wall 223 may be connected to the outer wall 224 via a bridge portion 235 as illustrated in, for example, FIG. 9.

Guide member body 220 further includes one or more shoulders 232 between a main region 231 and a leading region 233 of channel 230. When present, one or more shoulders 232 defined by the inner wall 223 may take on any suitable form which presents an abutment feature. As depicted, for example in FIG. 6, shoulders 232 taper towards the leading region 233 of the channel 230.

As shown in, for example FIG. 9, a cross-sectional dimension of leading region 233 of the channel 230 is less than a cross-sectional dimension of the main region 231 of the channel 230. The relatively narrow cross-sectional dimensions of the leading region 233 of the channel 230 contributes to the removal of excess faecal sample from the sample capture end 73 of the sampling member 70 as discussed further below.

While not shown, leading region 233 may include one or more restriction regions being regions of reduced diameter of the channel. The restriction regions may include a number of features of the inner wall 223 including, but not limited to, ridges or bumps which extend into the channel. In one embodiment, the inner wall includes one or more inwardly protruding annular rims. The restriction regions are adapted to assist to meter the faecal sample as sampling member 70 is withdrawn through the region.

Sampling device 210 is particularly suited to obtaining a faecal sample from the rectum of a subject, although it should be understood that sampling device 210 may also be suitable for obtaining other sample types from a subject via a different cavity.

As shown in for example FIGS. 2 and 4A and 5A, sampling assembly 90 comprises sampling device 10 and an elongate sampling member 70.

Sampling member 70 comprises a shaft 71 extending from a handling end 72 to a sample capture end 73.

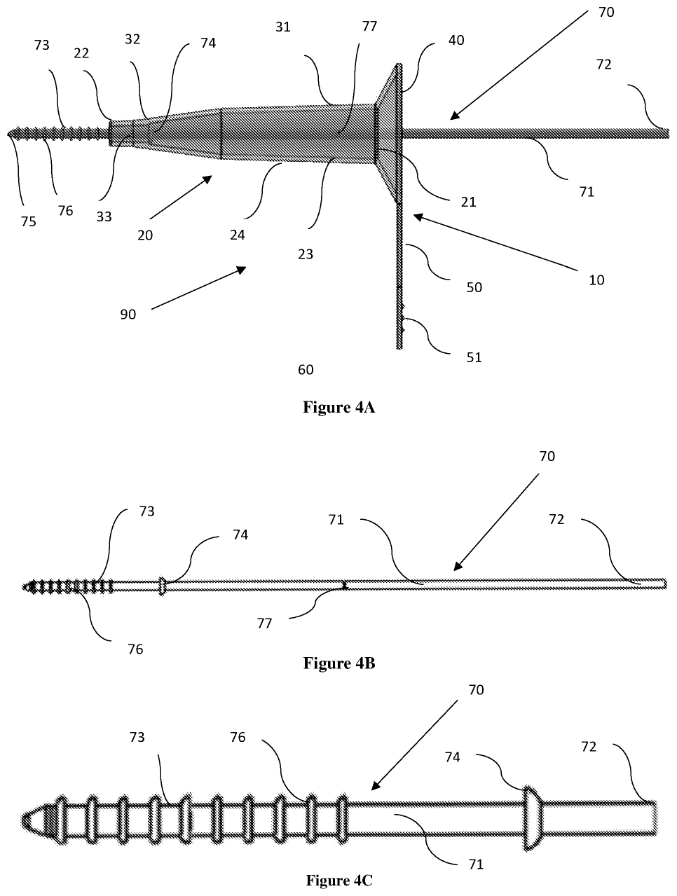

Sampling member 70 may further comprise a collar 74 as shown in FIG. 3 and FIGS. 4A to 4C. Where present, collar 74 is adapted to abut the one of more shoulders 32 of the guide member body 20 when the sampling member 70 is inserted into guide member body 20.

In the embodiment shown in, for example, FIGS. 2, 4B and 5B sampling member 70 further comprises one or more breakpoints 77 which allow the user to easily decrease the length of sampling member 70 as required. Breakpoints 77 may be molded or provided, for example, by way of a small cut.

Shaft 71 may be of a uniform cross sectional dimension, especially in the region of sample capture end 73, to facilitate wiping the sample capture end 73 as it is withdrawn through opening 25, 225 and channel 30, 230. Alternatively, shaft 71 may taper, or taper in sections. For example, shaft 71 may taper from handling end 72 providing a thicker shaft to allow for easier grip of handling end 72. Handling end 72 may also include a finger grip as shown in FIG. 5B.

The absorptive capacity, materials and/or dimensions of sample capture end 73 may be specifically selected to absorb the amount of stool moisture that gives the required test sensitivity. As shown in, for example, FIGS. 2 and 3, sampling member 70 optionally comprises a cellulosic material or matrix 80 at sample capture end 73. The cellulosic material or matrix 80 may be wound around sample capture end 73. The cellulosic material or matrix may comprise positively charged (cationic) materials or compounds. The positively charged materials may be non-woven and the positively charged materials and/or compounds may be hydrophilic.

In an embodiment, as shown in FIGS. 4A to 4C, sampling member 70 may comprise one or more capture features 76 disposed along and/or around shaft 71 at sample capture end 73. Capture features 76 are configured to capture a test sample, such as faecal matter, while minimising the risk of damaging the mucosal wall of the anal cavity of a subject. Accordingly, capture features 76 may, for example, comprise protrusions, ridges, ribs, microcollars or inwardly curved hooks.

As shown, for example in FIG. 5A, the dimensions of the sampling member 70 are such that only the capture end 73 extends beyond opening 25, 225.

Sampling member 70 may further comprise additional features as desired such as label features or features which aid in the storage, transport or safe disposal of the sampling member.

It should be understood that the sampling member 70 may also be used without the use of sampling device 10, or 210.

Devices 10, or 210 may be used to obtain a faecal sample. In use, guide member body 20, or 220 is inserted into the rectum of a subject. The guide member body 20 may be fully inserted until outer surface 43 of flange 40 abuts with the skin around the anus thus preventing further insertion. Guide member 220 may be inserted until collar 240 abuts with the skin around the anus. The guide member body 20, 220 need not, however, be inserted this far.

Sampling member 70 may then be inserted into open end 21 of guide member body 20 and advanced through channel 30 until sample capture end 73 of the sampling member 70 extends beyond opening 25.

Similarly, sampling member 70 may be inserted into open end 21 or 221 and advanced through channel 30, 230. Where present, one or more shoulders may provide an abutment surface for collar 74 of sampling member 70.

Where present, cap, seal or plug 60, 260 may be displaced or perforated by sampling member 70 as it is pushed through opening 25, 225 and into the rectum of the subject. That is, cap, seal or plug 60, 260 is moved from the first covering configuration to the second released configuration by advancement of sampling member 70. Cap, seal or plug 60, 260 may remain attached to guide member body or it may be displaced into the rectum or anal cavity of the patient wherein cap, seal or plug 60, 260 may later be reabsorbed or expelled.

Once sample capture end 73 of sampling member 70 is pushed beyond opening 25, 225 into the rectum, a faecal sample may be captured. The user may manoeuvre sampling member 70 around inside the rectum of the subject which may assist in the capture of a sufficient quantity of faecal sample. For example, the user may gently move sampling member 70 in a proximal-distal direction, a side-to-side direction, or in, for example, a twirling motion. Such motion may assist in loading sample capture end 73 with a faecal sample.

It is possible that after guide member body 20 or 220 is inserted via the anal canal into the rectum that guide member body 20 or 220 may then be retracted partially, for example by a distance of 5 mm or 10 mm or 15 mm or 20 mm before the sampling member 70 is pushed through the guide member and into the rectum. This allows the rectal canal to be dilated before the sampling member 70 is inserted, which reduces the likelihood that the insertion of the sampling member 70 may scratch the internal rectal wall tissues and possibly cause bleeding, which would contaminate the sample.

Sampling member 70 is then withdrawn from the anal cavity through opening 25, 225 and leading region 33, 233 of the channel 30. In doing so, the relatively larger cross-sectional part of the sample capture end 73 is withdrawn through the relatively smaller opening 25, 225 and the leading region 33, 233 of channel 30, 230 which removes any excess faecal sample from the sample capture end 73. Where present, sample capture end 73 may also be withdrawn through restriction regions of the leading region 33, 233 wherein the restriction regions further assist to meter the faecal sample.

Sample capture end 73 of sampling member 70 is configured, in conjunction with opening 25, 225, leading region 33, 233, and optionally restriction regions to collect a metered faecal sample.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A sampling device comprising:
an elongate guide member body having an open end and a leading end;
the guide member body having an inner wall which defines a channel extending from an opening in the leading end to the open end;
the inner wall defining:
a main region of the channel, the main region extending from the open end towards the leading end;
a leading region of the channel; and
one or more shoulders between the main region of the channel and the leading region of the channel;
a flange member extending from the open end of the guide member body.

2. The sampling device of claim 1, further comprising a flange member extending radially outwardly from the guide member body to an outer flange rim.

3. The sampling device of claim 2, comprising a handle extending from the outer rim of the flange member.

4. The sampling device of claim 1, wherein the guide member body further comprises a cap to cover or seal the opening in the leading end.

5. The sampling device of claim 4, wherein the cap comprises a plug configured to sit at least partially within the leading region of the channel.

6. The sampling device of claim 1, wherein an external surface of the guide member body is smooth and devoid of sharp edges, corners or protrusions.

7. The sampling device of claim 1, wherein a shoulder defined by the inner wall provides an abutment surface.

8. The sampling device of claim 7, wherein the abutment surface comprises an angled surface of the inner wall extending from a junction with the inner wall which defines the main region of the channel to a junction with the inner wall which defines the leading region of the channel.

9. The sampling device of claim 1, wherein a cross sectional area of the leading region of the channel is less than the cross sectional area of the main region of the channel.

10. The sampling device of claim 1, wherein the inner wall defining the leading region of the channel includes one or more restriction regions of reduced diameter of the channel.

11. The sampling device of claim 10, wherein the one or more restriction regions comprises a substantially annular rim which extends from the inner wall into the channel.

12. The sampling device of claim 1, wherein the guide member body has a length from the leading end to the open end of between 30 mm and 120 mm.

13. The sampling device of claim 1, wherein the length of the leading region of the channel is between 1 mm and 15 mm.

14. A sampling assembly including a sampling device comprising an elongate guide member body having an open end and a leading end;
the guide member body having an inner wall which defines a channel extending from an opening in the leading end to the open end;
the inner wall defining:
a main region of the channel, the main region extending from the open end towards the leading end;
a leading region of the channel; and
one or more shoulders between the main region of the channel and the leading region of the channel; and
a sampling member including a shaft extending from a handling end to a sample capture end, the sampling member configured to extend through the channel of the guide member.

15. The sampling assembly of claim 14, wherein the sampling member further comprises a collar.

16. The sampling assembly of claim 14, wherein the sampling member comprises one or more protrusions or ridges disposed along the shaft at the sample capture end.

17. The sampling assembly of claim 14, wherein the sampling member comprises a flocked material at the sample capture end.

18. The sampling assembly of claim 14, wherein the sampling member comprises a cellulosic material at the sample capture end.

19. The sampling assembly of claim 18, wherein the cellulosic material comprises positively charged materials or compounds.

20. A method for obtaining a faecal sample from a subject, the method comprising:
inserting a sampling device comprising an elongate guide member body into the anal cavity of a subject, the guide member body comprising:
an open end and a leading end;
an inner wall which defines a channel extending from an opening in the leading end to the open end, the inner wall defining:
a main region of the channel, the main region extending from the open end towards the leading end;
a leading region of the channel; and
one or more shoulders disposed between the main region of the channel and the leading region of the channel;
inserting a sampling member into the open end of the guide member, the sampling member having a shaft, a handling end and a sample capture end wherein at least part of the sample capture end has a cross-sectional area greater than the cross-sectional area of the shaft;
advancing the sampling member through the channel until the sample capture end extends beyond the opening in the leading end of the guide member body;
capturing a faecal sample on the sample capture end of the sampling member;
withdrawing the sampling member, wherein withdrawal of the sample capture end through the opening and the leading region channel of the guide member body removes any excess faecal sample from the sample capture end; and
further withdrawing the sampling member through the main region of the channel and from the open end of the guide member body.

21. The method of claim 20, wherein advancing the sampling member beyond the opening in the leading end of the guide member body includes perforating a cover across the opening.

22. A method of testing a faecal sample, the method comprising:
the method of obtaining a faecal sample from a subject according to claim 20; and
testing the faecal sample for the presence of blood, including by detection of blood components such as haemoglobin, globin or subunits thereof or other indicators of lower intestinal pathology, including genomic markers or intestinal pathogens.
